(19) United States
(12) Patent Application Publication
(10) Pub. No.: US 2007/0148215 A1
(43) Pub. Date: Jun. 28, 2007

(54) Therapeutically active dressings, their manufacture and use

(75) Inventors: Alexander Teslenko, Hagen (DE); Boris Alekseevich Nikonow, S-Petersburg (RU); Sergej Fedorovich Antonow, Nikolskoye (RU)

Correspondence Address: WILLIAM COLLARD;COLLARD & ROE, P.C., 1077 NORTHERN BOULEVARD, ROSLYN, NY, 11576, US

(73) Assignee: SANGUIBIOTECH GMBH, 58455 WITTEN GERMANY (DE)

(21) Appl. No.: 10/584,387

(22) PCT Filed: Nov. 30, 2004
(86) PCT No.: PCT/EP04/13575

§ 371 Date: Nov. 14, 2006

(30) Foreign Application Priority Data
Dec. 26, 2003 (RU) 2003138256

(51) Int. Cl. A61L 15/00

(52) U.S. Cl. 424/445

(57) ABSTRACT

The invention relates to therapeutically active wound dressings based on polysaccharides, in particular chitosan, and protein, in particular collagen/gelatine, with improved properties, their manufacture, in particular the use of polycarbonic acids and polyfunctional amino acids, and their use, especially in the medical field.

### OBJECT OF THE INVENTION

The invention relates to therapeutically active dressings based on polysaccharides, in particular chitosan, and protein, in particular collagen/gelatine, with improved properties, their manufacture, in particular the use of polycarbonic acids as well as preferably polyfunctional amino acids, and joint polymer dialysis, as well as their use, especially in the medical field.

### STATE OF THE ART

The number of patients with chronic, poorly-healing wounds is on an upward trend, despite enormous advances in medicine. There are various well-known chemical and pharmaceutical preparations in use in the local treatment of wounds. These are available in various pharmaceutical forms such as ointments, gels, plasters, films, powders etc. It is clear from the diversity of wound-healing preparations on the market today that there is no one universal preparation. Local wound treatment entails keeping harmful factors away from a wound and supporting mechanisms that promote healing. The following parameters are important in the development of a medical device such as this: antibacterial effect, wound-healing effect, and last but not least ease of handling e.g. detaches easily from the wound etc.

Regardless of the type of wound and the extent of tissue loss, every wound healing process undergoes three overlapping but inseparable phases: inflamemation, prolixferation and modulation. The immune system is of central importance in wound healing. Immune-competent cells are integrated within the mesenchymal cells of the new granulation tissue and direct a complex network from cells of the extracellular matrix and cell mediators.

Biologically active substances and pharmacological substances are indispensable in the effective treatment of wounds. Wounds in the first healing phase are painful and hard for a patient to bear. The inflammatory processes responsible for pain are dependent on a great number of factors, including active oxygen radicals, so-called ROS—reactive oxygen species (hydroxyl-radicals and superoxide-anions). Therefore it is assumed that when the level of radicals in the wound goes down, the pain intensity will also be reduced at the same time. It is well-known that chitosan can trap free radicals (Park P. J., et al. J. Agric. Food Chem. 2003, 51 (16): 4624-7; Je J Y, et al. Food Chem. Toxicol. 2004, 42 (3): 381-7). However, it is not sufficiently effective. The neutralization process of ROS can be considerably boosted by the addition of superoxide dismutase (SOD) and catalase to the wound dressing. Dismutase breaks down the superoxide anion and catalase (breaks down) hydrogen peroxide: both are harmful reactive oxygen substances. Even in the latest technology there are no dressings to date that contain SOD and catalase.

Moreover, it is well-known that retinoids and vitamin A play an important role in the epithelialization and contraction of wounds. The use of retinoids in the treatment of various skin conditions is well-known. It has been proven that retinoids play an important role in the biosynthesis and catabolism of collagen. They prevent the expression of collagenase and increase the expression of metalloprotease inhibittors in human fibroblasts (Bizot-Foulon V. et al. Cell Biol. Int. 1995.19 (2): 129-35). It is also known that a high dose of retinoids causes undesirable effects on the skin (erythema, desquamation and dermatitis).

Including retinoids in liposomes can significantly increase biotolerability and at the same time prevent rapid inactivation (Bizot-Foulon V. et al. J. Cosmetic Sci. 1998, 20 (2): 343-354). However, no dressings have been described to date using substances such as this.

Chronic wounds such as diabetic foot, decubitus (ulcers) or wounds due to venous or arterial insufficiency are caused by oxygen deficiency (hypoxia). The level of oxygen partial pressure in the wound is a determining parameter in the amputation of an organ. It is well-known that the amino acid arginine can prevent the risk of vasculopathy (U.S. Pat. No. 5,359,007, 2002). It has been shown on experimentally-induced diabetic wounds in animals that arginine improves wound healing and increases the rate of collagen biosynthesis (Shi H. P., et al. Wound Repair Regener. 2003. 11(3): 198-203).

For example, the amino acid taurine displays antioxidative activity (Franconi F. et al. Neurochem. Res. 2004, 29(1): 143-150), and stimulates cell proliferation and collagen biosynthesis. Taurine is able to trap the monochloramines, that have arisen through the neutralization of hypochlorides, and hence improve the wound healing process (Kato S., et al. Aliment. Pharmacol. Therap., 2002, 16 (2): 35-43). Thus arginine, taurine and other polyfunctional amino acids are without doubt hugely significant in the development of pharmacologically active dressings. In addition, they play an important role in the wound as building blocks in the biosynthesis of collagen and other proteins and glycoproteins.

Dressings are indispensable in the care of wounds today. As a provisional tissue matrix, an effective wound dressing should fulfill a number of functions: apart from protecting the wound from negative environmental influences (e.g. bacterial infections) as a covering material, good water and gas exchange, good sorption capacity for water and toxins (e.g. endotoxins or inflammation mediators), a wound dressing should also serve if possible as a framework (as a replacement for a natural extracellular matrix) for the new cell growth, and at least positively influence cell growth based on its own biological activity or biological agents present. In addition, it should be able to serve as a depot for the therapeutic preparations already mentioned above.

Hydrophilic wound dressings are particularly useful as porous materials (U.S. Pat. No. 4,572,906, 1986; U.S. Pat. No. 4,570,696, 1986; U.S. Pat. No. 4,659,700, 1987; U.S. Pat. No. 4,956,350, 1990; U.S. Pat. No. 5,169,630, 1992; U.S. Pat. No. 5,324,508, 1994; U.S. Pat. No. 5,871,985, 1999; U.S. Pat. No. 6,509,039, 2003; U.S. Pat. No. 6,608,040, 2003, RU 2007180, 1994; RU 2028158, 1996, RU 2193895,2002) and in particular as sponges and membranes.

The problem with these materials (membranes and films) is that they only have small pores that are too small for the migration of cells (fibroblasts, keratinocytes etc.) and do not permit three-dimensional growth of granulation tissue. In addition, owing to its low porosity, the material is not able to adsorb large quantities of exsudate from the wound.

In contrast, porous sponges have better properties (U.S. Pat. No. 5,116,824,1992; US 2002161440, 2002; DE 101 17234 A1, 2002).

Endogenous collagen plays an important role, especially in the wound healing process. After cleavage by collagenase, released collagen degradation products cause the migration and activation of inflammatory cells e.g. macrophages and hence influence the healing process at an early stage. This is why there are a lot of wound dressings and hemostatic sponges that are based on collagen and gelatine: Drop Collagen® (Master Aid), AngioSeal® (Wright Medical Biomaterial Products), Nobakoll® (NOBA), PolyPly® (Royce Medical), Matrix Collagene® (Collagen matrix, Inc.), Suprasorb C® (Lohmann & Rauscher GmbH). However, their principal function is a passive physical one, i.e. to cover the wound and adsorb exsudate. Hence the development of wound dressings containing a therapeutically active substance. We are aware of dressings based on collagen B (RU 561564, 1965), chitin, gelatine and formaldehyde (CH1097980, 1995), gelatine and formaldehyde with antibiotic (RU 2033149, 1995), cellulose with chitosan (JP 0376029, 1990), collagen and chitosan (PCT 8504413,1986), collagen and chitosan with antibiotic (RU 96124444,1998). However, the disadvantage of dressings such as this is that they are not very effective when it comes to wound healing. This is due to the wound adhering to the dressing and, in the case of collagen sponges, to an inadequate supply of oxygen to the wound.

It is also well-known that collagen displays the best biotolerability in the wound healing process as it has a natural fibrillary structure (U.S. Pat. No. 4,378,017, 1983). The quarternary biopolymer structures including proteins are stabilized by a hydrate shell to polar groups of polymers. If there is no such interaction between water and polar groups of biopolymers, the structure of the biopolymer is impaired when water is removed (drying process). Some low-molecular substances, so-called cosmotropic agents, are able to stabilize the hydrate shell present and hence to maintain the macromolecular structures during the drying process and afterwards. Research is being carried out into stabilizers such as this and their applications (Crow L. M. et al. Interaction of sugar with membranes: Biochim. et Biophys. Acta, 1988, V.947, 367-384. Carpenter J. F. et al. The mechanism of cryoprotection of proteins by solutes; Cryobiology 1988, V.25, 244-255). Monosaccharides and polysaccharides are traditionally used in the freeze-drying of proteins. It has been shown that these are not suitable in the manufacture of sponges according to the invention.

The effectiveness of wound dressings and membranes such as this based on collagen and gelatine was considerably improved by the admixture of biotolerable polymers e.g. oxycellulose: Promogran® (Johnson & Johnson), alginic acid: Fibracol® (Johnson & Johnson) or mucopolysaccharide: Catrix® (Lescander, Inc.) etc. Non-toxic, biotolerable chitosan is particularly interesting because of its special properties: the cationic polysaccharide chitosan forms ionic complexes with anionic molecules and polymers. It is used in the immobilization of a number of therapeutic and biologically active substances, e.g. the immobilization of proteins and microorganisms or in the binding of bacterial endotoxins (Davidova VN et al., Biochemistry (RU), 2000, 65 (9), 1082-90).

The heterogenic polysaccharide chitosan is composed of N-acetyl-D-glucosamine and D-glucosamine and is chemically similar to glucosaminoglycans in the skin. This endows chitosan with a number of interesting biological properties (e.g. macrophage activation, immunostimulation etc. in Khor E. “Chitin. Fulfilling a Biomaterials Promise. Elsevier, Amsterdam, 2001).

It has been proven in a number of studies that it is the breakdown products of chitin/chitosan, i.e. chito-oligosaccharides (COS), and not the products themselves that are biologically active/Muzzarelli R.A.A.(ed) Chitosan per os, from dietary supplement to drug carrier AtecEdizioni. 2000./. According to current understanding, the enzymatic activity of hydrolytic enzymes, such as chitinase, lysozyme or hexoaminidase, play a critical role in the context of human physiology. Even macrophages are able to produce a large number of these enzymes in the human body/Muzzarelli R.A.A. Human enzymatic activities related to the therapeutic administration of chitin derivates. CMLS. Cellular Molec. Life Sci. 1997, 53: 131-140.

As already mentioned, the unique properties of chitosan in conjunction with other polymers e.g. gelatine or collagen are hugely significant in the development of pharmaceutically active wound dressings and bioprostheses. In addition to the materials already mentioned, the following materials are well-known in specialist circles: chitosan/collagen sponges and chitosan/gelatine sponges (U.S. Pat. No. 4,659,700, 1987; U.S. Pat. No. 5,166,187, 1992; U.S. Pat. No. 5,116,824, 1992; U.S. Pat. No. 5,836,970, 1998; U.S. Pat. No. 5,871,985, 1999; US 2002161440, 2002; U.S. Pat. No. 6,565,878, 2003). In particular, the following patents relating to chitosan-collagen-based dressings can be mentioned in this connection: U.S. Pat. No. 5,116,824, 1992; U.S. Pat. No. 5,166,187, 1992, U.S. Pat. No. 5,836,970, 1998; US 2002161440, 2002; U.S. Pat. No. 6,565,878, 2003; RU 8608 B, 1998. In the patents cited, chitosan is dissolved in acetic acid, which later becomes neutralized. One serious disadvantage of this process is that heterogeneous solutions are formed. After water is removed, sponges form that have an undesirable morphology, i.e. their porosity is low and their pore shape and size is not optimal for cell growth. This affects their effectiveness in practical application. Materials such as this only absorb 1000 to 2000% of their own weight. One disadvantage of the technologies described in the patents cited above is that a high proportion of waste is produced (up to 70%). Lyophilization makes sponges contract, with an associated loss of porous structures. Furthermore, the wound dressing has an uneven surface and therefore does not cover the wound properly. It has also been established that acetic acid residues in the dressing can irritate the wound. Therefore, in order to remove the excess acetic acid, polymer solutions have been intensively dialyzed for up to six days. However, even after this treatment, acetic acid residues could still be detected.

### PURPOSE OF THE PRESENT INVENTION

Based on these findings, the purpose of the present invention is to develop a new, pharmacologically active wound dressing based on polysaccharides and proteins, which will have improved therapeutic properties. In particular it will contain active substances such as SOD, other enzymes and cytokins, polyfunctional amino acids, and also liposomal-encapsulated retinol, and be able to incorporate excipients. The stability of the proteins and the porosity of the wound dressings will be such as to allow improved water absorption and greater effectiveness, i.e. the above-mentioned disadvantages will be overcome. Furthermore, the dressing will be effective on different types of wound and in different stages of healing and therefore be more universally applicable.

### SOLVING THE STATED PROBLEMS

According to the invention, a wound dressing will be manufactured using a new process, which will be based on structural polysaccharides such as chitosan and structural proteins in particular, especially collagen and/or gelatine, which is characterized by a proportion of proteins, especially collagen, gelatine, derivatives or mixtures thereof, chitosan or derivates thereof, as well as polycarbonic acids. It will preferably also contain polyfunctional amino acids, if necessary a proportion of active substances and the rest will be made up of excipients and/or additives. The wound dressing will additionally be cross-linked. Surprisingly, this will help to overcome the disadvantages described above by application of polyfunctional amino acids, and primarily by avoiding the monocarbonic acid, acetic acid, as explained below.

Examples 1 to 9 document the manufacture and properties of the dressings according to the invention, also in comparison with known products.

Examples 10 and 11 verify the improved effectiveness of the dressings according to the invention.

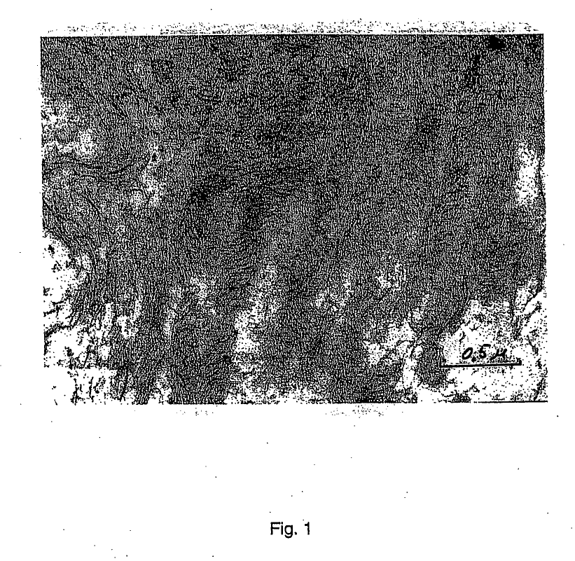

FIG. 1 shows the formation of fibrils in a collagen-chitosan mixture manufactured using the latest technology (RU 8608, acetic acid);

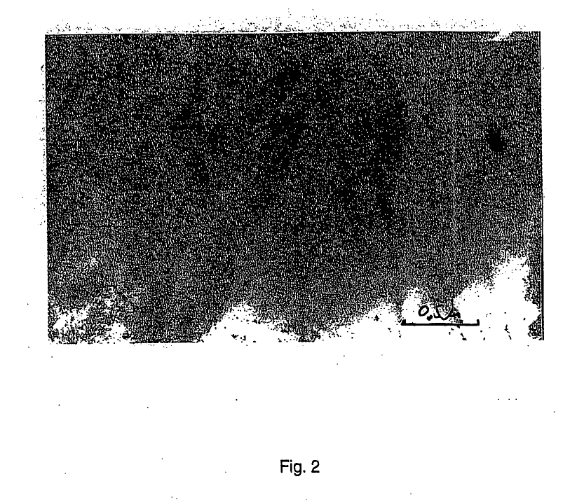

FIG. 2 shows the improved formation of fibrils in a collagen-chitosan mixture manufactured according to the invention using polycarbonic acid.

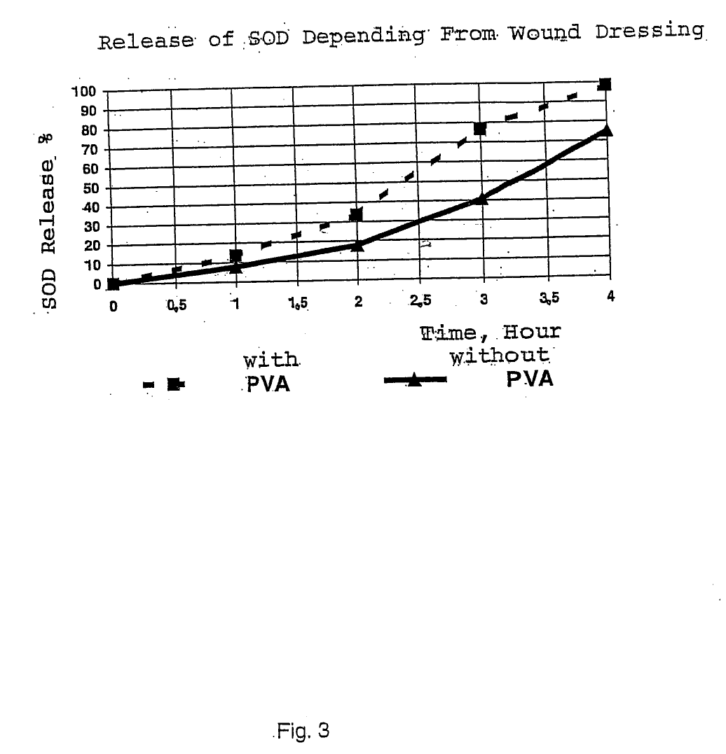

FIG. 3 shows the additional improvement in wound dressings due to the effect of excipients.

### BRIEF EXPLANATION OF DIAGRAMS

FIGS. 1 and 2 show the formation of fibrils in a collagen-chitosan mixture, in one using acetic acid in accordance with RU 8608 (FIG. 1) and in the other using polycarbonic acid (invention—FIG. 2). FIG. 3 shows the prolonged effect of superoxide dismutase in a wound dressing with and without polyvinyl alcohol (PVA).

### MORE DETAILED EXPLANATION OF THE INVENTION

The wound dressing comprises 19 to 56% structural proteins, in particular collagen, gelatine, derivatives or mixtures thereof, 18 to 58% structural polysaccharides, in particular chitosan or derivatives thereof, 0.5 to 10% polycarbonic acids, 0 to 15% but preferably 0.1 to 15% polyfunctional amino acids, 0 to 10% active substances and 0 to 30% additives and/or excipients in addition to 0.1 to 5% cross-linking agents. Primarily it also has a water-absorption capacity of over 2000%, primarily 2500 to 10000%, preferably 3000 to 7000%. It has a porous (sponge) structure. In its particularly advantageous developments, it has a specific weight of 0.01 to 0.06 g/cm³.

Chitosan is especially preferred in this respect as it has a mean molecular weight of greater than 200 to 500 kD, collagen type I and III and gelatine type A or B.

Polycarbonic acids according to the present invention are understood to be carbonic acids that in addition to a carboxyl group contain one or more functional groups (hydroxy-, carboxy-, amino acids etc.). Suitable polycarbonic acids include lactic acid, malic acid, succinic acid, pyrrolidone carbonic acid, malonic acid, fumaric acid, ascorbic acid, glutaminic acid, salicylic acid etc. or mixtures thereof. Preferable polycarbonic acids include lactic acid, succinic acid and others from the citrate cycle.

Preferable polyfunctional amino acids include arginine, methionine, proline, glutamic acid, alanine, taurine, glycine cysteine, n-acetylcysteine or mixtures thereof. Active substances chosen include in particular superoxide dismutase and/or catalase of various origin, primarily in a concentration of 0.001 to 1.0% to the polymer base. Alternatively or in addition, it may contain the pharmacologically active substance β-carotene of various origin, especially in liposomal form, where β-carotene is preferably used in a concentration of 0.001 to 0.5% to the polymer base.

Suitable additives include in particular antibacterial substances such as chlorhexidine, PolySept, polihexanide or suitable derivatives thereof. These may preferably be used in a concentration of 0.01 to 0.6% to the polymer base.

Excipients are selected in a preferred embodiment from plasticizing agents such as glycerine, or high-molecular substances, that guarantee adhesion to the wound surface or substances that affect the elimination of pharmaceutically active substances, or mixtures thereof. Excipient quantities of 10-30% are particularly suitable. In an advantageous embodiment glycerine and other polyols are used, especially polyvinyl alcohol and polyvinyl pyrrolidone.

The cross-linking agent chosen is a bifunctional cross-linking agent, preferably in a quantity of 0.1 to 5%, primarily glutaraldehyde.

The wound dressings according to the invention are manufactured according to a new process, which considerably increases the yield of products (sponges) of a suitable structure.

The manufacturing process. is as follows:
1. Instead of acetic acid, polycarbonic acids, including acids belonging to the citrate cycle (i.e. biologically active), are used (Tables 1 to 4). In particular, it is further preferred that
2. Instead of neutralization, a dialysis process takes place, that leads to the formation of optimal protein structures (e.g. collagen fibres).
3. Surprisingly, polyfunctional amino acids can also be used. On the one hand, these stabilize the quartenary structures of protein compounds. They play an important role as cryoprotectors and in the formation of pores. On the other hand, they display their own biological activity and hence accelerate the wound healing process.
   This is inconsistent with current understanding, which proposes monosaccharides and disaccharides as the usual stabilizers. These have been proven unsuitable in the manufacture of sponges according to the invention. Surprisingly, therefore, these substances do not need to be used, hence avoiding their undesirable effects on the wound.
4. Surprisingly, SOD, other enzymes, β-carotene, other provitamins and other pharmacologically active substances can also be used.
5. Surprisingly, antiseptics and excipients can also be used.

The manufacturing process according to the invention consists in dissolving the structural polysaccharide, in particular chitosan, and the structural protein, in particular collagen, gelatine or collagen-gelatine mixtures, each separately with polycarbonic acids, which may be the same or different, in water and then mixing these together and dialyzing them. Then, preferably cryoprotectors and structure formers, selected from polyfunctional amino acids, and additives are added, and the resultant gel freeze-dried.

The polycarbonic acids are preferably used in a ratio of 1:4 to 2:1 based on dry weight and the amino acids in a concentration of 0.1-15% (see table 3).

The following polycarbonic acids are primarily used in order to obtain optimal biotolerability: lactic acid, malonic acid, succinic acid, fumaric acid or mixtures thereof as components of the citrate cycle. Mixtures of the polysaccharide, in particular chitosan and protein, in particular collagen (gelatine) solutions are mixed and in particular maturated for a minimum of 12 hours and then dialyzed.

The dialysis process is broken off when the pH of the solution reaches 5.2-6.5. The dialysis process has an enormous influence on the availability of the pharmaceutically active substances (e.g. SOD) from the finished dressing into the wound (see Table 5.). Therefore, it is preferable if the ratio of polymer solution to water is at least 1:20, and in particular 1:50 to 1:200 over a period of 16 to 24 hours. The volume ratio of polymer solutions to water during dialysis can be 1:100 in a particularly preferred development of the invention (see table 5).

Polyfunctional amino acids may be contained in the wound dressing as cryoprotectors, pore formers and biologically active substances in an optimal concentration of 0.1 to 15% each.

SOD and catalase primarily are the active substances used. These are contained in an optimal quantity of 0.1 to 0.25 mg/cm² and reduce the inflammatory process in the wound and hence the associated pain. The use of retinoids causes acceleration of the wound healing process and epithelialization. Granulation phase.

Additives used include antibacterial substances e.g. chlorhexidine bigluconate and/or PolySept (up to 0.6%), excipients from the polyalcohol group e.g. glycerine (10-30%), high-molecular substances to improve adhesion to the wound, and to influence the elimination of pharmaceutically active substances e.g. polyvinyl alcohol and/or polyvinyl pyrrolidone (4-10%) (Table 4).

Certified starting materials that contain no toxic or infectious components e.g. viruses or prions, should be used in the manufacture of wound dressings. Certified type I and III collagens from a company called Belkosin (Russia) and gelatins manufactured according to the Pharmacopoeia (PB Gelatins, Gelita Europe, Rousselot a Sobel Company) and chitosan (Sonat and Bioprogress (Russia), Hydagen® (Cognis AG), Chitosan (Protan, Inc.) can be used.

It is an advantage if one can choose the starting material, especially in the case of chitosan. Chitosan is obtained from chitin by deacetylation and depolymerisation. Chitin with a deacetylation grade of greater than 85% is used for medical purposes. Chitosan is heterogeneous as far as its molecular weight is concerned. This can vary between 20 and 1000 kDa, depending on the manufacturing process. We have shown that chitosan with a molecular weight of less than 100 kDa shows a cytotoxic effect in fibroblast culture on chitosan films. This does not lead to cell death but a monolayer is unable to form on the surface of the chitosan. The cultured fibroblasts have an unusual morphology. In this case, the low-molecular chitosan is able to penetrate the cells and react with the proteins of the cytoskeleton or cytoplasm.

On the other hand, chitosan with a molecular mass of greater than 250 kDa and glucosamine and chitooligosaccharide with a molecular mass of less than 10 kDa, display no cytotoxic effects.

A similar dependency was also shown for intestinal flora. Low-molecular chitosan displays bacteriostatic, and in several cases, bactericidal properties.

According to the invention, for a biotolerable wound dressing chitosan with a molecular weight of greater than 200 kDa should preferably be used.

The dialysis according to the invention (electrodialysis) then removes the low-molecular chitosan. In addition, excess polycarbonic acids and other low-molecular impurities are removed from the starting materials during dialysis. As far as collagen is concerned, it has been discovered that during dialysis according to the invention a fibrillary collagen structure is formed. Surprisingly, with gelatine, dialysis increases the weight of spiral structures (increase in optical activity).

Dialysis is performed according to the composition of the polymer mixture. The pH should be between 5.5 and 6.5. To achieve this pH, only a single short-term dialysis process is necessary for the gelatine-chitosan mixture. A larger quantity of water may be needed for a collagen-chitosan solution.

According to the invention, a mixture of polymer solutions is used during dialysis. Surprisingly, this produces an improvement in the porous sponge structure. In contrast, dialysis of separate polymer solutions, each containing one polymer, leads to a heterogeneous pore structure and uneven wound dressing surface.

According to RU 8608 B, 1998, it is well-known that dialysis against water of a collagen-chitosan mixture in acetic acid leads to the self-generation of collagen fibrils, which is very heavily influenced by the presence of chitosan in the solution. Surprisingly, this effect is considerably boosted by the use of polycarbonic acids as in the invention, which are not acetic acids. FIGS. 1 and 2 show the formation of fibrils in a collagen-chitosan mixture, according to RU 8608 (acetic acid—FIG. 1) and according to polycarbonic acid (invention—FIG. 2). It can clearly be seen that exchanging acetic acid for polycarbonic acid boosts the formation of fibrils. This is evident in the significantly higher electronic density of the fibrillary structure.

In preferred embodiments a particularly effective self-organization of collagen fibrils takes place during dialysis at a ratio of polymer solution to water of approx. 1 to 100 over a duration of 16 to 24 hours.

Excipients used can be low-molecular compounds such as glycerine and in particular high-molecular polymers (polyvinyl alcohol, polyvinylpyrrolidone etc.). Surprisingly it has been shown that the wound dressing structures are even more positively influenced and consequently are more effective during use (e.g. adhesion to wound surface). They are also able to prolong the effect of pharmaceutical preparations. FIG. 3 shows the prolonged effect of superoxide dismutase in wound dressings with and without polyvinyl alcohol (PVA).

In order to increase the stability of wound dressings to enzymes present in the wound, and to increase the time the dressing can be left on the wound, cross-linking agents are used that consist of the class of mono or bifunctional agents, or are based on physical methods. The use of cross-linking chitosan agents is described in detail in the literature. Glutaraldehyde (GA) is preferably used as it is an effective cross-linking agent. This choice is based on known findings that the residual concentration of GA during the manufacture of dressings is outside its cytotoxic effect, i.e. less than 0.004 p.p.m (Beyer K I, et al. Chitosan-Collagen-Sponges. Estimation of the residual concentration of crosslinking agent. p. 327-328. in. Proc. 4^{th} World Meeting ADRITELF, Florence 2002). It was shown, especially when GA is used in a concentration of 0.01% to 0.03% and at a pH of 5.2 to 6.2, that a three-dimensional cross-linked chitosan matrix can be formed. Free collagen fibrils or gelatine molecules can be found in this matrix.

According to the invention, manufacture of pharmaceutically active wound dressings consists of the following steps:
The required amounts of chitosan and collagen (gelatine) are dissolved in water together with polycarbonic acids (the same or different) (each separately).
The resultant polymer mixtures are dialyzed together.
After that, polyfunctional amino acids, pharmacological preparations and excipients and/or additives are added.
Cross-linking agents are added to the resultant solution.
The resultant gel is frozen at −20 to −60° C. and subsequently lyophilized.
The sponge material is packaged and sterilized.

The following examples 1 to 9 present preferred embodiments of the process according to the invention.

Application

The wound dressings according to the invention are applied by cutting the dried product into suitable tiles and placing them on the wounds.

Biotolerability and Pharmacological Effect of the Dressings According to the Invention (Preclinical Studies)

Preclinical studies on the wound dressings according to the invention have revealed no toxic, irritant or sensitizing effects. The wound dressings are apyrogenic, and according to their sanitary, hygienic and toxicological parameters they comply with the required safety standards for medical devices that come into contact with human wounds.

In experiments on rats with artificially induced burns, a better healing effect was shown using the wound dressing according to the invention than with alginate-based dressings.

In addition, an antibacterial effect was shown with the wound dressings according to the invention.

Clinical Studies

The wound dressings according to the invention were tested in randomized, controlled, blinded, multicenter clinical studies on patients with wounds of varying etiology. The wound dressings were placed on the wound and fixed in place with gauze or suitable equivalent. During absorption of wound exsudate there is a slow diffusion of biologically and pharmacologically active substances contained in the dressing into the wound. Through contact with the wound, chitosan stimulates repair processes and accelerates wound healing.

The biologically active substances and collagen fibrils stimulate cell activity e.g. the immune response. Parts of the dressing are slowly integrated into the wound and the remainder rejected after successful epithelialization.

If wounds were infected and very productive, the dressings were changed daily. If wounds are well-granulated, the dressing can remain on the wound until it has healed completely.

The clinical studies showed that the dressings were highly effective in the healing of infected and chronic wounds, burns and surgical wounds at the following phases of wound healing:
Inflammatory and cleaning phase
Granulation phase
Epithelialization phase

Based on these data, the wound dressing according to the invention is a universal wound dressing that can be used in all phases of wound healing. Compared with the usual methods, wound healing time is shortened by at least 20%.

A detailed description of the practical application of the wound dressings according to the invention is given in examples 10-11.

The dressings according to the invention are particularly suitable in the treatment of post-traumatic and surgical wounds, and in addition accelerate healing of first to third degree burns, and are especially suitable in the healing of infected or chronic wounds of varying etiology.

### EXAMPLES

This invention is not limited to the examples presented. It has a wider application.

### Example 1

3 g lyophilized collagen was added to 150 ml of an aqueous solution of 1% glutaminic acid and 1% malic acid. At the same time, 3 g chitosan was dissolved in 150 ml 1% succinic acid. The two solutions were mixed and dialyzed against water over the course of 20 hours until a pH of 5.2 was obtained. 0.04 g SOD, 0.03 g catalase and 0.06 ml 20% chlorhexidine bigluconate solution and 20 ml 3% arginine solution were added to this solution. Then 5 ml 1.25% glutaraldehyde solution was added and finally the resultant gel was freeze-dried.

The wound dressing in accordance with RU 8608 B, 1998 was prepared at the same time:

3 g lyophilized collagen was dissolved in 150 ml 3N acetic acid. 3 g chitosan was dissolved in 150 ml 2% acetic acid.

The two solutions were mixed and dialyzed over the course of 20 hours against water to a pH of 5.2.

0.04 g SOD and 0.06 ml 20% chlorhexidine bigluconate solution and 20 ml 3% arginine solution were added to this solution. Then 5 ml 1.25% glutaraldehyde solution was added and finally the resultant gel was freeze-dried.

The cells containing the frozen materials were treated in a freeze drier at −35° C. at a freezing rate of 4° C. , 10° C. und 20° C. per hour and subsequently dried.

In all cases, porous materials were obtained, which had the characteristics presented in tables 1 and 2 below.

TABLE 1
Comparison of the new wound dressing using the manufacturing
procedure according to the invention against a control.

| | | Sponge with |
|---|---|---|
| | Rate of freezing | unsuitable |
| Wound dressing | ° C./hour | structures, in % |
| (Control) | 4 | 33 |
| | 10 | 72 |
| | 20 | 100 |
| According to the invention | 4 | 0 |
| | 10 | 0 |
| | 20 | 2 |

TABLE 2
Comparison of wound dressings (from example 1) with homogenous (process according
to the invention) and non-homogenous porous structure (control).

| | | | | Wound dressings |
|---|---|---|---|---|
| | | | Wound dressings | with non- |
| | | Unit of | with homogenous | homogenous pore |
| Parameter | | measurement | pore structure | structure |
| 1. | Appearance | — | Dry, porous | Dry, porous |
| | | | structure, odorless, | structure, odorless, |
| | | | pale beige | pale beige |
| 2. | Elasticity | — | The sponge can be | The sponge breaks |
| | | | rolled up | when rolled up |
| 3. | Dimensions | mm | 75 × 50 × 10 (±2) | 60 × 40 × 8 (± 5) |
| 4. | Quantity of SOD | %, on dry | 7.4 ± 0.3 | 0.06 ± 0.04 |
| | released over 24 | weight | | |
| | hours | | | |
| 5. | Specific weight | g/cm³ | 0.014 ± 0.002 | 0.034 ± 0.015 |
| 6. | pH of aqueous | pH | 5.8 ± 0.2 | 5.8 ± 0.2 |
| | extract | | | |
| 8. | Residual moisture | % | 15.3 ± 0.5 | 38.2 ± 4.5 |
| 9. | Adsorption | % to dry weight | 7100 ± 500 | 1700 ± 1100 |
| | capacity (water) | | | |
| 10. | Gas permeability | mg/cm²/hour | 4.5 ± 0.5 | <0.1 |
| | of wound dressing | | | |

### Example 2

Example 2 shows the effect of composition and concentration of polycarbonic acids and amino acids on the properties of wound dressings. Table 1 shows how the wound dressings are prepared.

TABLE 3
Effect of manufacturing conditions on wound dressing properties

| | | | Chitosan/ | | | Properties | |
|---|---|---|---|---|---|---|---|
| | Carbonic acid | Amino acid | PCA- ratio in | PCA/AA-ratio in | | Brittelness | |
| No | (PCA) | (AA) | starting solution | wound dressing | Waste % | (hardness) | Porosity |
| 1 | Succinic acid | Glycine | 1:8 | 1:0.85 | 100 | high | large |
| | | (8.5%) | | | | | |
| 2 | Succinic acid | Glycine | 1:4 | 1:1.8 | <12 | high | large |
| | | (7.3%) | | | | | |
| 3 | Succinic acid | Glycine | 1:2 | 1:3.3 | <2 | low | large |
| | | (7.3%) | | | | | |
| 4 | Glutaminic acid | Glycine | 1:2 | 1:3.3 | 17 | low | medium |
| | | (7.3%) | | | | | |
| 5 | Malonic acid | Glycine | 1:2 | 1:3.3 | 6 | low | large |
| | | (7.3%) | | | | | |
| 6 | GAS | Glycine | 1:2 | 1:7.5 | 100 | high | large |
| | | (15%) | | | | | |
| 7 | GAS | Glycine | 1:2 | 1:3.3 | 0 | low | large |
| | | (7.2%) | | | | | |
| 8 | GAS | Glycine | 1:2 | 1:1.5 | <2% | low | medium |
| | | (3.2%) | | | | | |
| 9 | GAS | Glycine | 1:2 | 1:0.9 | >70% | high | none |
| | | (2.0%) | | | | | |
| 10 | Succinic acid | Glycine | 1:1 | 1:6 | <2% | low | large |
| | | (7.2%) | | | | | |
| 11 | Succinic acid | Glutamin | 1:1 | 1:6 | <5% | low | large |
| | | (7.2%) | | | | | |
| 12 | Succinic acid | Alanine | 1:1 | 1:6 | <3% | low | large |
| | | (6.6%) | | | | | |
| 13 | Succinic acid | Glycine | 1:0.5 | 1:15 | <10% | low | medium |
| | | (15%) | | | | | |
| 14 | Succinic acid | Glycine | 1:1 | 1:3.6 | 0% | low | medium |
| | | (7.2%) | | | | | |

GAS: Succinic acid and malic acid in 1:1 ratio by weight

TABLE 4
Composition (in wt %) of wound dressings with various
ratios of carbonic acid and amino acids.

| | No. corresponding to table 3 | | | | | | |
|---|---|---|---|---|---|---|---|
| Composition wt % | 1 | 2 | 3 | 4 | 5 | 6 | 7 |
| Chitosan | 57.0 | 36.4 | 44.3 | 44.3 | 36.8 | 35.8 | 18.0 |
| Collagen | 19.0 | 36.4 | 44.3 | 44.3 | 36.8 | 35.8 | 56.0 |
| Succinic acid* | 10.0 | 4.1 | 2.2 | | | | |
| Glutaminic acid* | | | | 2.2 | | | |
| Malonic acid* | | | | | 2.2 | | |
| Acid mix* | | | | | | 2.0 | 2.2 |
| Glutaraldehyde | 5.0 | 0.7 | 1.0 | 1.0 | 0.6 | 0.6 | 0.7 |
| SOD | 0.5 | 0.1 | 0.9 | 0.9 | 0.9 | 0.8 | 0.9 |
| Gebitan | | | | | | | |
| Glycerine | | 15.0 | | | 15.4 | 10 | 15.0 |
| Glycine | 8.5 | 7.3 | 7.3 | 7.3 | 7.3 | 15.0 | 7.2 |
| Glutamine | | | | | | | |
| Alanine | | | | | | | |
| Polyvinylalcohol | | | | | | | |
| | No. corresponding to table 3 | | | | | | |
| Composition wt % | 8 | 9 | 10 | 11 | 12 | 13 | 14 |
| Chitosan | 58.4 | 42.5 | 36.1 | 35.0 | 30.5 | 41.3 | 31.0 |
| Collagen | 35.4 | 42.5 | 36.1 | 35.0 | 30.5 | 41.3 | 31.0 |
| Succinic acid* | | | 1.2 | 1.2 | 1.1 | 1.0 | 2.1 |
| Glutaminic acid* | | | | | | | |
| Malonic acid* | | | | | | | |
| Acid mix* | 2.2 | 2.2 | | | | | 2.0 |
| Glutaraldehyde | 0.7 | 0.8 | 0.6 | 0.7 | 0.6 | 0.5 | 0.9 |
| SOD | 0.1 | 10 | 0.9 | 0.9 | 0.7 | 0.3 | 0.6 |
| Gebitan | | | | | | 0.6 | 0.2 |
| Glycerine | | | 17.9 | 20.0 | 30.0 | | 20 |
| Glycine | 3.2 | 2.0 | 7.2 | | | 15 | 7.2 |
| Glutamine | | | | 7.2 | | | |
| Alanine | | | | | 6.6 | | |
| Polyvinylalcohol | | | | | | | 5.0 |

*The residues of carbonic acid in the wound dressings were calculated in accordance with corresponding calibrations for acids in solutions

### Example 3

3 g lyophilized collagen was added to 150 ml of a solution of 2% succinic acid. At the same time, 3 g chitosan was dissolved in 150 ml 1% succinic acid. The two solutions were mixed and dialyzed against water over 20 hours until a pH of 5.2 was obtained. 0.04 g SOD or 0.03 g catalase and 20 ml 2% proline solution were added to this solution.

The resultant solution was divided into two equal parts. 2.5 ml 1.25% glutaraldehyde solution was added to one part. 0.3 g polyvinylalcohol in 20 ml water was added to the other part. Then 2.5 ml 1.25% glutaraldehyde solution was added. The resultant materials were frozen at −35° C. and freeze-dried. SOD in a concentration of 0.6% of the dry weight or 0.1 mg per cm² was detected in the prepared wound dressings. The specific density was 0.014+/−0.001 g per cm³. The water adsorption capacity was 7100+/−500%. The wound dressings containing polyvinyl alcohol had uniform and somewhat smaller pores. The elimination of SOD was determined after determining the activity of SOD by the delay in quercitine oxidation (Cao GH, et al. Methods Enzymol. 1990, 186, 161-168). The wound dressings containing polyvinyl alcohol showed a delayed elimination of SOD upon rehydratation in water (FIG. 3).

### Example 4

3 g chitosan and 3 g collagen were dissolved in 300 ml 2% malic acid. Then we proceeded as described in example 1. Then liposomes containing 15 ml carotene were added to the polymer solution. The liposomal β-carotene was prepared as described in the following section:

1.2 g phospholipids (Nattermann Phospholipid GmbH) and 0.3 g β-carotene were added to 10 ml chloroform. 8.4 g polyvinyl alcohol was added to the resultant solution. The resultant suspension was stirred thoroughly and poured into a cell. The chloroform was removed in a vacuum cupboard. The dry product was milled and then added to 150 ml phosphate buffer of pH 6.8 to 7.4 and stirred thoroughly for 30 minutes. This produced multi-layered liposomes. To make the liposomes smaller in size and at the same time sterilize them, they were treated with ultrasonic waves (150-200 W).

The liposomes containing 0.2% β-carotene and 2% phospholipids were added to the collagen-chitosan solution. To this were added 0.02 ml 20% chlorhexidine bigluconate and 0.006 ml PolySept and up to 30% plasticizing agent e.g. glycerine. The cross-linking and freeze-drying were carried out as described in example 1.

The finished product was cut, packaged and labeled. Then it was sterilized by gamma radiation at a dose of 1.5 Mrad.

Wound dressings containing carotene are primarily used to heal chronic wounds. A stimulating effect on the epithelialization process was detected in all studies performed.

### Example 5

3 g gelatine was allowed to steep in 150 ml 0.1% lactic acid and then dissolved at 70° C. 3 g chitosan with a molecular mass of 350 kDa and deacetylation grade of over 85%, and 0.7 g succinic acid were dissolved in 130 ml water until the chitosan had completely dissolved. The two solutions were mixed and then dialyzed against water in a ratio of 1:100. To this solution was added 20 ml 20% taurine solution. Cross-linking and subsequent freeze-drying were carried out as described in example 1. The sponges obtained have a specific density of 0.012 to 0.016 g/cm³. The water absorption capacity ranges from 4000 to 6000%.

### Example 6

10 g chitosan and 5 g lactic acid were added to 900 ml apyrogenic deionised water. The solution was stirred until the chitosan had completely dissolved.

0.9 g pyrrolidone carbonic acid and 10 g gelatine were added to 900 ml apyrogenic deionised water and dissolved by heating to 70° C.

The two solutions were mixed together and dialyzed. Then 40 ml 20% glycine solution was added. The cross-linking and freeze-drying were carried out as descrybed in example 1.

The sponges obtained have a specific density of 0.020 to 0.022 g/cm³. The water absorption capacity ranges from 4000 to 5000%.

The finished product was cut, packaged and labeled. Then it was sterilized by gamma radiation at a dose of 1.5 Mrad.

### Example 7

10 g chitosan was dissolved in 500 ml deionised water containing 10 g succinic acid.

10 g collagen was dissolved in 500 ml deionised water containing 10 g succinic acid.

The two solutions were mixed and subsequently dialyzed against water in a ratio of 1:100.

2 g gelatine was allowed to steep in 100 ml water and then dissolved at 70° C. The solution obtained was autoclaved at 0.5 bar for 45 minutes at 125° C. The autoclaved gelatine was then mixed with chitosan-collagen solution.

The amino acids and peptides obtained through thermohydrolysis are able to stabilize the polymer structures of the wound dressing.

0.1 ml chlorhexidine bigluconate was added to this solution. The cross-linking and freeze-drying were carried out as described in example 1.

The sponges obtained have a specific density of 0.012 to 0.016 g/cm³. The water absorption capacity ranges from 5000 to 8000%.

The finished product was cut, packaged and labeled. Then it was sterilized by gamma radiation at a dose of 1.5 Mrad.

### Example 8

10 g chitosan was dissolved in deionised water containing 10 g succinic acid.

10 g collagen was dissolved in 500 ml deionised water containing 10 g succinic acid.

The two solutions were mixed and subsequently dialyzed against water in varying ratios of water to polymer solution.

0.05 g SOD and 80 ml 2% glycine solution were added to this solution.

The cross-linking and freeze-drying were carried out as described in example 1.

Table 5 presents the characteristics of the manufactured products.

TABLE 5
Effect of dialysis on the properties of wound dressings.

| | Solution- | Duration of | pH of | | pH of | SOD activity |
|---|---|---|---|---|---|---|
| | water | dialysis, | starting | pH after | water | in % to |
| No | ratio | hours | solution | dialysis | extract | native SOD |
| 1 | 1:20 | 20 | 4.2 | 4.5 | 4.6 | 42 ± 10 |
| 2 | 1:50 | 20 | 4.2 | 4.7 | 4.9 | 68 ± 10 |
| 3 | 1:100 | 20 | 4.2 | 5.2 | 5.6 | 87 ± 10 |
| 4 | 1:100 | 10 | 4.2 | 4.7 | 4.8 | 63 ± 10 |
| 5 | 1:200 | 20 | 4.2 | 5.4 | 6.0 | 90 ± 10 |

The SOD activity was determined by inhibition of the quercitin reaction. Wound dressings that were prepared by dialysis in a ratio of 1:100 and 1:200 for 20 hours showed the highest SOD activity.

### Example 9

The polymer solutions were prepared as shown in example 8.

The wound dressings were prepared using two different procedures.

In one procedure, the chitosan and collagen solutions were mixed and subsequently dialyzed.

In the other procedure, the chitosan and collagen solutions were dialyzed separately to begin with and then mixed together.

Then 0.1 ml chlorhexidine bigluconate, 40 ml 2% arginine and 2 g glycerine were added to the solutions obtained.

The cross-linking and freeze-drying were carried out as described in example 1.

The resultant wound dressings differed in terms of appearance. The wound dressings resulting from the first procedure were elastic and had good porosity, whereas the wound dressings resulting from the second procedure were inelastic, polydisperse and had an uneven surface.

Clinical Testing of the Wound Dressing According to the Invention.

### Example 10

Sterile wound dressings, prepared as in example 1, were used for clinical testing. Clinical studies were carried out on 29 patients aged between 26 and 92 years of age to treat wounds of varying etiology (chronic wounds, decubitus (ulcers), post-traumatic wounds) in the granulation phase.

Before applying the wound dressings, the wounds were cleaned according to standard practice.

The packaging of the wound dressings was opened using sterile scissors. The wound dressings were then cut according to the size of the wound, allowing an excess of 0.5 cm. The wound dressing that was cut out was pressed down firmly onto the surface of the wound and also fixed in place using gauze or a suitable equivalent.

The wound dressings were soft from absorption of wound exsudate and adhered well to the wound. When chronic wounds were treated with wound dressings containing superoxide dismutase, the wound healing process appeared to proceed normally:

The surface of the wound was cleaned of fibrin residues and filled with pink finegrained granulation tissue.

In the epithelialization phase the wound dressing was kept on the wound until it had completely healed. After removing the bandage, the epithelized surface of the wound was free of hyperkeratosis (no large scars).

When treating post-traumatic wounds (following autoplasty), the wound dressings were applied to the wound immediately after the trauma had occurred. This caused a hemostatic effect. The wound dressings were kept on the wound until epithelialization was complete.

Compared with the control, an accelerated wound healing process was observed. Complete epithelialization occurred four to five days earlier with burns and post-traumatic wounds. In the case of chronic wounds, a 50% reduction in surface area and depth of wound was detected 10-15 days earlier than in the control (table 6). Application of the wound dressings according to the invention produced an approx. 30% reduction in requirement for dressings to be changed. This was also seen as a benefit by the patients.

Based on clinical observations, a stimulating effect on wound repair processes was shown, especially in elderly patients with a delayed healing process. No infection or deterioration in healing process was observed.

When the wound dressings according to the invention were used, the patients did not complain of any symptoms such as pain, itching or burning.

In addition, the wound dressings according to the invention were determined to have an effective antihypoxic effect. This antihypoxic effect is probably due to the presence of succinic acid and arginine, which are capable of normalizing the respiratory process in the tissue, regardless of oxygen supply.

A large number of unpleasant side effects (pain, burning and itching) were observed in the control, particularly in the first few minutes after (start of) treatment. In addition, the unpleasant acetic acid smell was perceived as disturbing.

TABLE 6
Effectiveness of wound healing after application of wound dressings and prototypes
according to the invention to patients with chronic and post-traumatic wounds.

| | | | Results | | | | | |
|---|---|---|---|---|---|---|---|---|
| | | | | Frequency of | | Tolerability | | |
| | Number of | Wound | Adhesion to | change of | First | of wound | End of | Side |
| Diagnosis | patients | dressing | surface | dressing | granulation | dressing | epithelialization | effects |
| A | 19 | According | +++ | Every 2-3 | On day | Pain 0 | On day | None |
| | | to the | | days | 3-5 | Burning 1 | 8-9 | |
| | | invention | | | | Itching 0 | | |
| | | Control | ++ | Every 2-3 | On day | Pain 2 | On day | Allergic |
| | | | | days | 4-7 | Burning 2 | 12-15 | dermatitis 1 |
| | | | | | | Itching 0 | | |
| B | 12 | According | ++ | Every 2 | On day | Pain 0 | 50% reduction | None |
| | | to the | | days | 5-6 | Burning 0 | in wound on day | |
| | | invention | | | | Itching 0 | 30-35 | |
| | | Control | ++ | Every 2 | On day | Pain 3 | On day | Allergic |
| | | | | days | 8-10 | Burning 2 | 40 | dermatitis 3 |
| | | | | | | Itching 3 | | |

A Post-traumatic wounds (including after autologous dermatoplasty), B Chronic wounds due to CVI

### Example 11

Healing Infected Wounds with Wound Dressings from Example 3

Clinical studies were carried out on twelve patients aged between 17 and 28 years of age with poorly healing wounds that were caused by suppurative and inflammatory processes (phlegmons, phlebitis etc.).

Wound dressings based on acetic acid as in example 1 were used as the control.

After removal of pus and necrotic tissue, wound dressings as in example 11 were applied and the dressings were changed daily.

Clinical observations were made over the first 10-15 days. After 5-7 days of using the wound dressings according to the invention, the surface of the wound was cleaned of fibrin residues and filled with pink, fine-grained granulation tissue.

Over the next 4-5 days the surface area of the wound reduced considerably and the defective tissue filled with fresh granulation tissue.

Island and border epithelialization were observed approx. three days earlier than in the control in eight patients.

Analysis of results and cytological observations has shown that application of wound dressings according to the invention led to stimulation of the leukocytic reaction (approx. 35% greater than control) and to acceleration of the granulation and epithelialization processes. No negative side effects or reactions were observed.

1-32. (canceled)

33. Wound dressing, wherein they display from 19 to 56% of one or more structural proteins, chosen from collagen, gelatine, derivatives or mixtures thereof, 18 to 58% of one or more structural polysaccharides, chosen from chitosan and (or) chitosan derivatives or mixtures thereof, 0.5 to 10% polycarbonic acids, 0.1 to 15% polyfunctional amino acids, 0 to 10% active substances, 0 to 30% excipients and/or additives, and 0.2 to 5% cross-linking agents.

34. Wound dressing according to claim 33, wherein the polycarbonic acid is chosen from: lactic acid, malic acid, succinic acid, malonic acid, fumaric acid, ascorbic acid, glutaminic acid, salicylic acid, pyrrolidone carbonic acid or mixtures thereof.

35. Wound dressing according to claim 33, wherein as the polyfunctional amino acid the following are present: arginine, methionine, proline, taurine, glycine, alanine, cysteine, N-acetyl cysteine or mixtures thereof.

36. Procedure for the production of a wound dressing, containing 19 to 56% of one or more structural proteins, chosen from collagen, gelatine, derivatives or mixtures thereof, 18 to 58% of one or more structural polysaccharides, chosen from chitosan and (or) chitosan derivatives or mixtures thereof, 0.5 to 10% polycarbonic acids, 0.1 to 15% polyfunctional amino acids, 0 to 10% active substances, 0.2 to 5% cross-linking agents, 0 to 30% excipients and/or additives, wherein to an aqueous solution of the polysaccharide a polycarbonic acid is added and to an aqueous solution of a structural protein is added the same or a different polycarbonic acid, subsequently both polymer solutions are dialyzed together and then polyfunctional amino acids and active substances, cross-linking agents, additives and excipients of the dialyzed reaction mixture are added if necessary.

37. Procedure according to claim 36, wherein collagen of various origin is used as the structural protein.

38. Procedure according to claim 37, wherein gelatine type A and type B are used as the structural protein.

39. Procedure according to claim 38, wherein high-molecular gelatine with a Bloom value of greater than 200 is used.

40. Procedure according to claim 36, wherein chitosan, its water-soluble derivatives or mixtures thereof are used as the polysaccharide.

41. Procedure according to claim 36, wherein chitosan with a molecular weight of greater than 200 kDa is used.

42. Procedure according to claim 36, wherein as the polycarbonic acid succinic acid, lactic acid, malic acid, malonic acid, fumaric acid, ascorbic acid, glutaminic acid, salicylic acid, pyrrolidone carbonic acid or their mixtures are used.

43. Procedure according to claim 36, wherein the ratio of polycarbonic acids to high-molecular substances used is 1:4 to 2:1.

44. Procedure according to claim 38, wherein the solutions of structural polysaccharides, in particular chitosan and structural proteins, are mixed together at least 12 hours before dialysis.

45. Procedure according to claim 36, wherein dialysis against water takes place in a volume ratio of polymer solution to water of at least 1:100 over the course of more than 16 hours.

46. Procedure according to claim 36, wherein polyfunctional amino acids are added to the dialysed solutions.

47. Procedure according to claim 36, wherein as polyfunctional amino acids arginine, proline, glutamate, taurine, glycine cysteine, N-acetylcysteine are used.

48. Procedure according to claim 47, wherein the polyfunctional amino acids are used in concentrations of 0.1-15%.

49. Procedure according to claim 36, glutaraldehyde is used as the bifunctional cross-linking agent.

50. Procedure according to claim 36, wherein as the pharmacologically active substance superoxide dismutase and/or catalase of various origin is used.

51. Procedure according to claim 50, wherein superoxide dismutase and/or catalase are used in a concentration of 0.001 to 0.1% to the polymer base.

52. Procedure according to claim 36, wherein as the pharmacologically active substance β-carotene of various origin is used.

53. Procedure according to claim 52, wherein β-carotene in liposomal form is used as the pharmacologically active substance.

54. Procedure according to claim 52, wherein β-carotene is used in a concentration of 0.001 to 0.05% to the polymer base.

55. Procedure according to claim 36, wherein as excipients antibacterial substances chosen from chlorhexidine, PolySept, polihexanide, plasticizers, high-molecular substances, that guarantee adhesion to the wound surface and/or excipients that influence the excretion of pharmaceutically active substances are used.

56. Procedure according to claim 55, wherein antibacterial substances are used in a concentration of 0.01 to 0.6% to the polymer base.

57. Procedure according to claim 55, wherein the additives/excipients are added to the dialysate in a concentration of 10-30%.

58. Procedure according to claim 57, wherein polyvinyl alcohol and polyvinylpyrrolidone are used as excipients.

59. Use of a wound dressing in accordance with claim 33 for the production of an agent for the accelerated healing of post-traumatic and surgical wounds.

60. Use of a wound dressing according to claim 33 for the production of an agent, wherein the healing of first third degree burns is accelerated.

61. Use of a wound dressing according to claim 33 for the production of an agent, wherein the healing of infected or chronic wounds of various etiology is accelerated.

62. Use of a wound dressing according to 33, for the accelerated healing of post-traumatic and surgical, infected, chronic wounds or burns.
